(19) United States
(12) Patent Application Publication
(10) Pub. No.: US 2010/0228251 A1
(43) Pub. Date: Sep. 9, 2010

(54) COAGULATION STENCIL AND APPLICATION DEVICE

(76) Inventor: Andreas Hörlle, Berlin (DE)

Correspondence Address: OSTROLENK FABER GERB & SOFFEN, 1180 AVENUE OF THE AMERICAS, NEW YORK, NY, 100368403, US

(21) Appl. No.: 12/669,275

(22) PCT Filed: Sep. 3, 2008
(86) PCT No.: PCT/EP2008/061645

§ 371 Date: Jan. 15, 2010

(30) Foreign Application Priority Data
Sep. 7, 2007 (DE) 10 2007 042 524.6

(51) Int. Cl. A61B 18/14; A61B 18/12

(52) U.S. Cl. 606/49

(57) ABSTRACT

The invention concerns a coagulation stencil for arranging a group of high frequency electrodes, comprising a plurality of arrangements of holes each comprising three mutually equally spaced receiving holes which extend substantially parallel and in which the high frequency electrodes can be inserted. To provide an improved coagulation stencil it is provided in accordance with the invention that the receiving holes are arranged in two rows extending in substantially radiating form away from a common receiving hole and the spacing of each receiving hole of one of the arrangements of holes relative to the nearest outside contour of the coagulation stencil is less than five times the diameter of the receiving hole.

The present invention concerns a coagulation stencil for arranging high frequency electrodes, comprising a plurality of arrangements of holes each comprising three mutually equally spaced receiving holes which extend substantially parallel and in which the high frequency electrodes can be inserted.

Electrosurgical and in particular electrothermal sclerosing of pathologically altered tissue, hereinafter referred to for brevity as tissue, by coagulation is a method which is known in medicine. That method is of particular interest for the therapy of organ tumours, for example liver tumours. For the sclerosing procedure one or more high frequency electrodes is or are placed in the tissue to be sclerosed, that is to say the tumour tissue, or in the immediate proximity thereof. A circuit is then closed so that, when using a monopolar electrode arrangement, an alternating current flows between the electrodes and a neutral electrode fixed to the body on the outside thereof. When using a bipolar electrode arrangement the current flows between the electrodes in the tissue themselves (in that case there must be at least two electrodes). Reference is made to a multipolar application when there are more than two electrodes in the tissue, with alternating current flowing between the electrodes.

The electrodes intended to be placed in the tissue are generally in the form of electrode needles. They have an electrically conducting cylindrical shank which, with the exception of one or more distal regions, referred to as the active regions of the electrode or, for brevity, active electrodes, is electrically insulated in relation to the surrounding tissue. The active electrodes in contrast are electrically connected to the body tissue.

A current flow is induced between the active electrodes and the neutral electrode or electrodes in the monopolar arrangement by means of a high frequency generator. It is possible to dispense with the neutral electrode in the alternative bipolar or multipolar arrangement. In that case the circuit is closed by way of a further active electrode, wherein the required active electrodes can be disposed in a coaxial arrangement, in the bipolar application, insulated from each other on the electrode needle or, in the multipolar application, on two separate electrode needles.

The ohmic tissue resistance which is a part of the complex tissue impedance results in conversion of the alternating current applied by way of the electrodes into Joulean heat. At temperatures between 50 and 100° C. massive denaturing of the body-specific proteins, coagulation, occurs, and that results in the area of tissue involved dying off. By virtue of the high level of current density around the active electrodes, heating occurs predominantly in the region of those electrodes so that local thermal tumour destruction is possible.

In order to increase the size of the ablation or coagulation regions in the multipolar application, an electrode arrangement comprising a plurality of and in particular three electrodes is used for example in tumour treatments. The electrodes of the electrode arrangement are distributed uniformly around the tumour in order to coagulate the tumour tissue as completely as possible. For uniform coagulation with three electrodes the spacings between the electrodes should be as equal as possible. To simplify such placement and to avoid errors use is made of coagulation stencils of the above-indicated kind. In that case the coagulation stencil is arranged on or over the tissue and then the electrodes are introduced into the receiving holes of the selected arrangement of holes and into the tissue.

US No 2006/0079885 A1 describes by way of example a coagulation stencil in which the receiving holes for the electrodes are arranged centrally around a central receiving means for a guide needle.

Further coagulation stencils are disclosed in WO 2005/009528 A1, the receiving holes of which have steps or inserts for limiting the depth of insertion of the electrodes.

Further coagulation stencils are described for example in US No 2002/111615 A1, US No 2004/181216 A1,US No 2004/039429 A1, US No 2002/120261 A1 and U.S. Pat. No. 6,506,189 B1.

The known coagulation stencils suffer from the disadvantage that the gradation of the electrode spacings between the different arrangements of holes is relatively great so that it is not always possible to set the spacing which is optimum for the application involved. There is also the problem that the coagulation stencils for example impede an ultrasound sensor for positional checking of the electrodes and thus make positional checking difficult or even entirely prevent it.

Therefore the object of the present invention is to provide an improved coagulation stencil which resolves the problems found in the state of the art.

That object is attained by a coagulation stencil of the above-indicated kind in which the receiving holes are arranged in two rows extending in substantially radiating form away from a common receiving hole and the spacing of each receiving hole of one of the arrangements of holes relative to the nearest outside contour of the coagulation stencil is less than five times the diameter of the receiving hole.

The coagulation stencil according to the invention has the advantage that all the arrangements of holes provided, of the three receiving holes, jointly use a receiving hole. That makes it possible to have a smaller gradation between the arrangements of holes because the other receiving holes of the arrangements of holes are arranged in radiating form from the common receiving hole. Accordingly the invention overcomes the disadvantage of the coagulation stencils in the state of the art, in which for example gradations in the arrangements of holes in 5 mm steps are not possible as the receiving holes would overlap or would butt against each other.

In addition positional checking of the electrodes by means of ultrasound is more easily possible because the ultrasound sensor can be positioned close to the electrodes by virtue of the small spacing of the receiving holes relative to the outside contour.

The invention can be further developed by various advantageous configurations which are independent of each other.

Thus the coagulation stencil can be of an acute-angled configuration with two limbs, the rows of receiving holes can be arranged in the limbs and the limbs can extend substantially in the direction of the rows of receiving holes. The rows extend in particular at an acute angle, at an angle of 60° relative to each other. That has the advantage that the electrodes arranged in the receiving holes, in the region of the limbs, are easily accessible from two sides, for example for an ultrasound sensor. The surface area of a projection of the coagulation stencil according to the invention in the direction of the receiving holes can thus be smaller than the surface area of a coagulation stencil in the form of an equilateral triangle. The surface area can thus be smaller than

$$\frac{\sqrt{3} }{4} \cdot {s}^{2} ,$$

wherein s is the side length of the coagulation stencil. Preferably the surface area A of the coagulation stencil in the direction of the receiving holes can be smaller than

$$\frac{\sqrt{3}}{4} \cdot {( {a}_{max} + 6 D )}^{2} ,$$

wherein aₘₐₓ is the centre spacing of the largest arrangement of holes and D is the diameter of the receiving holes.

In order to prevent positioning of the electrodes in incorrect receiving holes the coagulation stencil can have markings by which the receiving holes of the individual arrangements of holes can be identified and clearly distinguished from each other. The markings can be the form of digits which specify the spacing of the receiving holes of the respective arrangement of holes. Additionally or alternatively the markings can form at least portion-wise equilateral triangles, wherein the corners of the triangles fall into the centre points of the receiving holes of the respective arrangement of holes.

In order further to improve accessibility for an ultrasound sensor for positional checking of the electrodes the spacing of each receiving hole relative to the nearest outside contour of the puncturing stencil can be less than three times the diameter of the receiving hole. In the case of usual configurations the diameter of the receiving hole can be less than 5 mm, in particular 3.5 mm or 2 mm.

Besides the coagulation stencil in the above-described embodiments the invention further concerns an application device for applying a high frequency current for thermal sclerosing of body tissue. The application device has an electrode arrangement comprising at least three high frequency electrodes which can be introduced into body tissue, a high frequency generator electrically connected to the high frequency electrodes for generating electrical high frequency energy and a coagulation stencil according to one of the aforementioned embodiments.

Further advantages and features of the present invention are described by means of detailed embodiments by way of example with reference to the accompanying drawings in which:

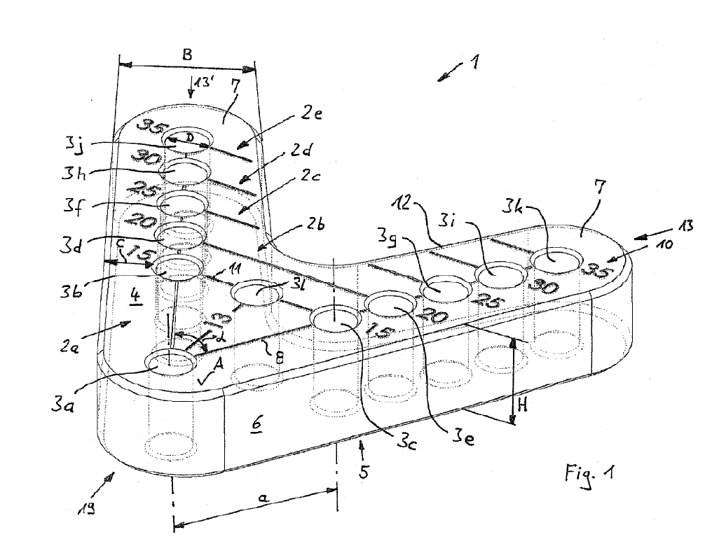

FIG. 1 shows a diagrammatic view of a first embodiment of a coagulation stencil according to the invention,

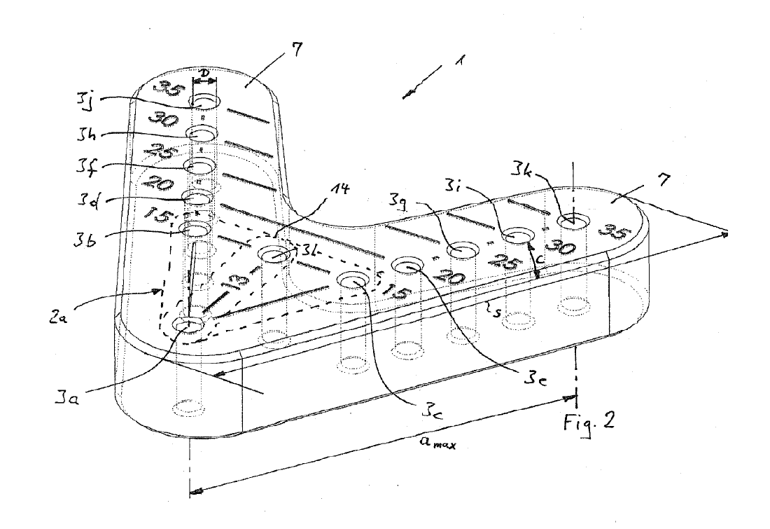

FIG. 2 shows a diagrammatic view of a second embodiment of a coagulation stencil according to the invention with a smaller diameter for the receiving holes,

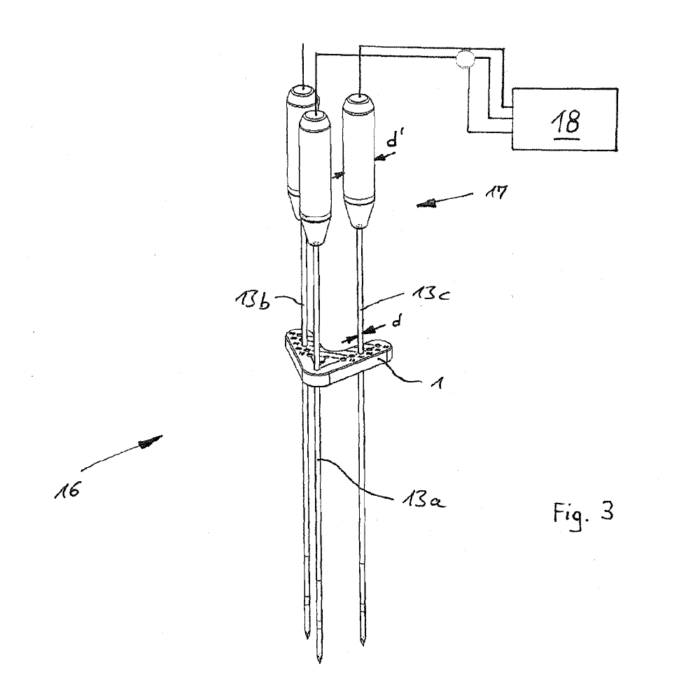

FIG. 3 shows a diagrammatic view of an application device according to the invention with the coagulation stencil of FIG. 2, and

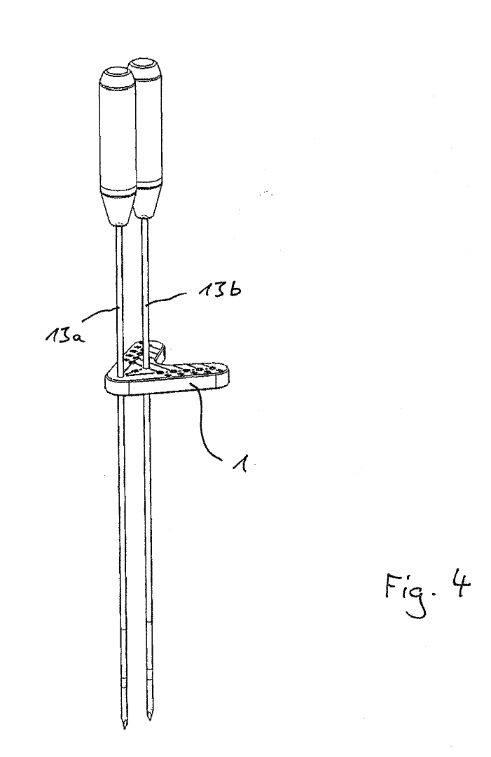

FIG. 4 shows a diagrammatic view of the coagulation stencil of FIG. 2 with two HF electrodes.

The invention will firstly be described by reference to the coagulation stencils 1 in FIGS. 1 and 2. The two embodiments by way of example of the coagulation stencil 1 according to the invention differ only by virtue of a differing diameter D for the receiving holes 3.

The coagulation stencil 1 has a plurality of arrangements 2 of holes each comprising three receiving holes 3 extending in parallel relationship. The coagulation stencils 1 only differ by virtue of the diameter D of the receiving holes 3, which is 3.5 mm in the embodiment of FIG. 1 and 2.2 mm in the embodiment of FIG. 2.

The coagulation stencil 1 has a substantially flat top side 4 and an underside 5 extending parallel to the top side 4. A side surface 6 of the coagulation stencil 1 extends substantially perpendicularly to the top side 4 and the underside 5.

In a plan view the coagulation stencil 1 is of an acute-angled configuration with two limbs 7. The two limbs 7 diverge at an angle α from a common receiving hole 3a arranged in the tip region 19. In the embodiments of FIGS. 1 and 2 the angle α is about 60°.

The coagulation stencil 1 is rounded off in the tip region 19, the transition between the limbs 7, and at the free ends of the limbs 7.

In both limbs 7 the other receiving holes 3b, 3d, 3f, 3h, 3j and 3c, 3e, 3g, 3i and 3k respectively are arranged one behind the other. The centre points of the receiving holes 3 are each disposed on a respective straight line 8 through the centre point of the receiving hole 3a. Accordingly the receiving holes 3 extend in radiating relationship in two rows 13′ away from the receiving hole 3a. The two rows 13′ and the straight lines 8 extend at the angle α relative to each other, like the limbs 7. The limbs 7 are of a width B, with the receiving holes 3 being arranged substantially in the centre of the limbs 7.

The coagulation stencil 1 is so shaped that a spacing C from one of the receiving holes 3 of the arrangements 2 of holes to the nearest outside contour 12 is less than five times and preferably three times the diameter D of the receiving holes 3. The outside contour 12, at the limbs 7, extends substantially parallel to the rows 13′ of the receiving holes 3.

The coagulation stencil 1 shown by way of example in FIG. 1 has five different arrangements of holes 2a, 2b, 2c, 2d and 2e, each comprising three receiving holes 3a, 3b, 3c and 3a, 3d, 3e and 3a, 3f, 3g and 3a, 3h, 3i and 3a, 3j, 3k respectively. The receiving holes 3 of the various arrangements 2 of holes are each equally spaced from each other and therefore form the corners of equilateral triangles. In the embodiment in FIG. 1 the centre spacing a of the receiving holes 3 in the case of the arrangement 2a of holes is 15 mm, in the case of the arrangement 2b of holes it is 20 mm, in the case of the arrangement 2c of holes it is 25 mm, in the case of the arrangement 2d of holes it is 30 mm and in the case of the arrangement of holes 2e it is 35 mm. The arrangements 2 of holes thus increase in size in 5 mm steps.

At the top side 4 the coagulation stencil 1 has various markings 9. The markings 9 identify the different arrangements of holes 2. Thus disposed beside the receiving holes 3b-3k are respective digits 10 which specify the spacing between the holes of the respective arrangement of holes in millimetres. In addition, equilateral triangles are marked, the corner points of which form the centre points of the receiving holes 3 of the respective arrangement 2. The markings 9 mean that the operator can readily see which receiving holes 3 belong together. In the embodiment of FIG. 1 the markings 9 are engraved in the top side 4. It will be appreciated that alternatively the markings 9 can also be provided in any other usual way, for example printed or raised.

Besides the receiving holes 3a-3k of the triple hole arrangements 2 the coagulation stencils 1 in FIGS. 1 and 2 have a further receiving hole 3l arranged in the centre between the receiving holes 3b and 3c. The further receiving hole 3l, with the receiving hole 3a, forms a double hole arrangement 14, in FIGS. 1 and 2 involving a spacing of 13 mm.

FIG. 2 shows an application device 16 according to the invention with an electrode arrangement 17 comprising three high frequency electrodes 13, an HF generator 18 connected to the high frequency electrodes 13 for generating a high frequency voltage and the coagulation stencil 1 of FIG. 2. The HF electrodes 13 are inserted into the receiving holes 3a, 3b and 3c of the arrangement 2a of holes. As the receiving holes 3 extend in substantially mutually parallel relationship and the diameter D of the receiving holes 3 is approximately equal to the outside diameter d of the high frequency electrodes 13 the inserted high frequency electrodes 13 are oriented in substantially mutually parallel relationship. Due to a relatively large thickness H for the coagulation stencil 1 the receiving holes 3 are of a sufficient guide length to guide the high frequency electrodes 13 in substantially mutually parallel relationship. The thickness H of the coagulation stencils 1 in FIGS. 1 and 2 is more than 6 mm, preferably more than 8 mm.

In order to increase the spacing of the three high frequency electrodes 13a to 13c from the 15 mm shown in FIG. 3, the high frequency electrodes 13b and 13c only have to be transposed into one of the larger arrangements 2b to 2e of holes. The high frequency electrode 13a does not have to be displaced because it is disposed in the receiving hole 3a which belongs to all arrangements 2 of holes. That common receiving hole 3a and the radiating arrangement of the other receiving holes 3b, 3d, 3f, 3h, 3j and 3c, 3e, 3g, 3i, 3k provides that 5 mm gradations between the arrangements 2 of holes are possible, with only two rows 13′.

FIG. 4 shows the coagulation stencil 1 according to the invention as illustrated in FIG. 2 in use with two high frequency electrodes 3 inserted into the receiving holes 3a and 3l of the double hole arrangement 14. With the double hole arrangement 14 two HF electrodes 13 can be arranged at a minimal spacing relative to each other. The minimal spacing is predetermined by the outside diameter d′ of the handles 15 of the HF electrodes 13. The handles 15 predetermine the minimal spacing at which they bear against each other in the double hole arrangement 14. It will be appreciated that further double hole arrangements with larger spacings are possible with the receiving hole 3a and one of the receiving holes 3b to 3k.

Depending on the respective size of the area to be coagulated, which depends for example on the size of tumour, two HF electrodes 13 are used in a double hole arrangement 14 or three HF electrodes 13 are used in a triple hole arrangement 2.

Due to the relatively small spacing C of the receiving holes 3 from the outside contour 12 accessibility to the inserted HF electrodes 13, for example with an ultrasound sensor, is improved. Positional control of the HF electrodes 13 can thus be better implemented, as is generally usual.

By virtue of the configuration of the coagulation stencil 1 with the two limbs 7, the area occupied is reduced in comparison with a configuration in the form of an equilateral triangle without the two limbs 7. Therefore the surface area A of the coagulation stencil 1 is smaller than

$$\frac{\sqrt{3}}{4} \cdot {s}^{2} ,$$

wherein s is the side length of the coagulation stencil. In the embodiments shown in FIG. 1 or FIG. 2 the surface area A is even smaller than

$$\frac{\sqrt{3}}{4} \cdot {( {a}_{max} + 6 D )}^{2} ,$$

wherein aₘₐₓ is the centre spacing of the largest arrangement 2e of holes and D is the diameter of the receiving holes 3. The surface area being reduced in relation to the state of the art means that there is an improvement in accessibility, for example for an ultrasound sensor, from both sides of the limbs 7, to the receiving holes 3.

1. A coagulation stencil for arranging high frequency electrodes, comprising a plurality of arrangements of holes each comprising three mutually equally spaced receiving holes which extend substantially parallel and in which the high frequency electrodes can be inserted, wherein the receiving holes are arranged in two rows extending in substantially radiating form away from a common receiving hole and the spacing of each receiving hole of one of the arrangements of holes relative to the nearest outside contour of the coagulation stencil is less than five times the diameter of the receiving hole.

2. A coagulation stencil according to claim 1 wherein the coagulation stencil is of an acute-angled configuration with two limbs, the rows of receiving holes are arranged in the limbs and the limbs extend substantially in the direction of the rows of receiving holes.

3. A coagulation stencil according to claim 1 wherein the coagulation stencil has markings by which the receiving holes of the individual arrangements of holes are identified and can be distinguished from each other.

4. A coagulation stencil according to claim 3 wherein the markings are in the form of digits which specify the spacing of the receiving holes of the respective arrangement of holes.

5. A coagulation stencil according to claim 3 wherein the markings form at least portion-wise equilateral triangles, wherein the corners of the triangles fall into the receiving holes of the respective arrangement of holes.

6. A coagulation stencil according to claim 1 wherein the surface area A of the coagulation stencil in the direction of the receiving holes is smaller than
$$\frac{\sqrt{3}}{4} \cdot {( {a}_{max} + 6 D )}^{2} ,$$
wherein aₘₐₓ is the centre spacing of the largest arrangement of holes and D is the diameter of the receiving holes.

7. A coagulation stencil according to claim 1 wherein the spacing of each receiving hole of one of the arrangements of holes relative to the nearest outside contour of the puncturing stencil is less than three times the diameter of the receiving hole.

8. A coagulation stencil according to claim 1 wherein the diameter of the receiving hole is less than 5 mm.

9. An application device for applying a high frequency current for thermal sclerosing of body tissue, comprising an electrode arrangement comprising at least three high frequency electrodes which can be introduced into body tissue, a high frequency generator electrically connected to the high frequency electrodes for generating electrical high frequency energy and a coagulation stencil according to one of the preceding claims in which the high frequency electrodes can be inserted.

10. A coagulation stencil according to claim 2 wherein the coagulation stencil has markings by which the receiving holes of the individual arrangements of holes are identified and can be distinguished from each other.

11. A coagulation stencil according to claim 10 wherein the markings are in the form of digits which specify the spacing of the receiving holes of the respective arrangement of holes.

12. A coagulation stencil according to claim 10 wherein the markings form at least portion-wise equilateral triangles, wherein the corners of the triangles fall into the receiving holes of the respective arrangement of holes.

13. A coagulation stencil according to claim 4 wherein the markings form at least portion-wise equilateral triangles, wherein the corners of the triangles fall into the receiving holes of the respective arrangement of holes.

14. A coagulation stencil according to claim 11 wherein the markings form at least portion-wise equilateral triangles, wherein the corners of the triangles fall into the receiving holes of the respective arrangement of holes.
